(19) United States
(12) Patent Application Publication
(10) Pub. No.: US 2006/0272109 A1
(43) Pub. Date: Dec. 7, 2006

(54) Method to apply a hair care preparation and kit for same

(76) Inventors: Ruth Stern, Toronto (CA); Pauline Ashworth, Toronto (CA)

Correspondence Address: BERESKIN AND PARR, 40 KING STREET WEST, BOX 401, TORONTO, ON, M5H 3Y2, CA

(21) Appl. No.: 11/371,944

(22) Filed: Mar. 10, 2006

Related U.S. Application Data
(63) Continuation-in-part of application No. 11/145,068, filed on Jun. 6, 2005.

(51) Int. Cl. A61K 8/18

(52) U.S. Cl. 8/406; 132/208

(57) ABSTRACT

A method for coloring hair with a hair care formulation that is mixed prior to use is provided. The method includes mixing together the components of the hair care formulation as the formulation is dispensed from an applicator having at least one storage compartment for each component. A test amount of the hair care formulation is mixed as it is dispensed and the applicator sealed while the formulation is applied to the user's skin to determine if the skin of the user reacts to the formulation. Subsequently, the applicator may be unsealed and an additional amount of the formulation is dispensed to treat the hair of the user. A kit for use in conducting the method is also provided.

### FIELD OF THE INVENTION

This invention relates to a method of treating hair. In a preferred aspect, the application relates to a method of coloring hair. In another preferred aspect, this invention relates to a kit for use in coloring hair.

### BACKGROUND OF THE INVENTION

The most common method of dyeing human hair is oxidative dyeing. Various formulations for use in oxidative hair dyeing or coloring are well known in the art. Such formulations typically involve the use of a two-part system. One part, the dye component, contains at least one primary intermediate and at least one coupler. For example, the dye component may contain diaminobenzenes, dihydroxybenzenes and aminophenols. Before use, this dye component is mixed with a second part, which is a developer formulation containing an oxidizing agent, such as hydrogen peroxide or other strong oxidizing agent. The developer oxidizes the primary intermediate (e.g. the benzenes), which then reacts with the coupler (e.g. the aminophenols) to form a colored compound. See for example U.S. Pat. No. 6,565,615 (Wong et al).

Typically, the dye component and the developer are mixed shortly prior to use. If a person has their hair dyed in a hair salon, then the hair coloring formulation is typically mixed by a hairdresser just prior to the application of the hair coloring formulation. Home hair coloring formulations are sold to the general public. Typically, these must also be mixed prior to use and, accordingly, an individual consumer must mix these formulations prior to use. Such formulations may be used to dye all or part of the hair of the person.

A common problem with using long-lasting hair dyes is that as a person's hair grows, the new hair that emerges from the scalp is uncolored. Accordingly, in some cases, the person will mix a hair coloring formulation to color the new hair instead of re-coloring all of their hair.

Hair coloring formulations may irritate the scalp of a user. Accordingly, it is typically recommended that a part of the hair coloring formulation be applied to the skin of a user up to 48 hours prior to the use of the formulation to color the person's hair to test whether the hair coloring formulation will not irritate the scalp. Accordingly, at least a part of the formulation must be decanted from its container and mixed so that the formulation must be tested on the person's skin. Typically, hair-coloring formulations must be utilized within about 45 minutes of being mixed. Therefore, a portion of the formulation must be decanted and mixed while the rest is stored for subsequent decanting and mixing, provided that the skin of the person does not react too much to the formulation.

Various types of containers for storing two components of a mixture are known in the art. These include, for example, U.S. Pat. Nos. 2,819,723; 3,570,499; 5,725,499 and 6,036,005.

U.S. Pat. No. 5,725,499 (Silverstein et al) discloses a dual barreled syringe, which may be used for storing and dispensing two components of a formulation. The formulation may be mixed once dispensed from the syringe or as it is dispensed from the syringe dual barreled syringe. It is known that a dual barreled syringe may be used to dispense a hair coloring formulation.

U.S. Pat. No. 2,819,723 (Mayer) and U.S. Pat. No. 3,570,499 (Ruta) disclose applicators for applying a hair treatment preparation. The applicators have two different compartments, which store different components of the hair treatment formulation. The different components are mixed as they are dispensed from the applicators. The applicators include comb or brush portions so that, once the hair treatment formulation is mixed, it may be brushed into the hair of a person.

U.S. Pat. No. 5,339,839 (Forcelledo et al) and U.S. Pat. No. 6,260,557 (Yarbrugh) disclose combs, which have passages extending from a base portion to the tip of the teeth of the comb, so that a hair coloring formulation may be passed through the comb and dispensed at the teeth of the comb.

A disadvantage with these devices is that the brush or comb portion of the applicator will have the hair coloring formulation stored therein while the person conducts a test to determine if the formulation will irritate their skin. Accordingly, if the formulation is not storage stable, the applicator (including the brush/comb portion) must be discarded and a new applicator used to color the hair following the successful testing of the formulation.

U.S. Pat. No. 6,036,005 (Krause et al) discloses a package for storing, mixing and dispensing a multi-component product, such as hair colorings (column 9, line 57). The package of Krause et al includes an outer container for holding a first material and an inner container, and an inner container (interior of the outer container), for holding a second material. When the outer container is squeezed, the inner container is opened and the first and second materials may be mixed together. One disadvantage with the package of Krause et al is that all of the hair coloring formulation would be mixed at once. Accordingly, a user would have to mix all of the formulation, apply a portion of the formulation to their skin to determine if it irritates their skin and subsequently use the remainder of the formulation to color their hair if the formulation does not irritate their skin. However, if the formulation, once mixed, is not stable during this test period, then the remainder of the formulation must be thrown out and a new package opened and used to color the person's hair.

### SUMMARY OF THE INVENTION

The method and kit of the instant invention utilize an applicator having two storage compartments, wherein each compartment contains a different component of a hair treatment formulation and, preferably, a hair coloring formulation and, most preferably, an oxidative hair dye. The hair coloring formulation may be any formulation, which is a two-component system that requires mixing prior to application to the hair. The hair coloring formulation may be used to color the hair of the user, or merely to tint, streak or highlight hair.

The applicator may be any applicator known in the art that stores the two components separately. The applicator is provided with a mixer nozzle. In operation, the mixer nozzle is connected in flow communication with each compartment of the applicator so that the two components enter the mixer nozzle and are combined together as they flow through the mixer nozzle. The outlet of the mixer nozzle therefore delivers a formulation wherein the two components have been mixed together. Such mixer nozzles are known in the art and any such mixer nozzle may be used in accordance with the instant invention. Preferably, the applicator is a dual barrel syringe, such as that of Sylverstein et al, that is provided with a mixer nozzle. It will be appreciated that the method and kit of the instant invention may be used with a formulation that comprises more than two components. In such an embodiment, the applicator will have more than two compartments. For example, if the formulation is a three-component system, then the applicator will have three components. It will be appreciated that each component may be provided in only one compartment or that a particular component may be provided in two or more compartments provided that the different components are stored in separate compartments.

In accordance to one aspect of this invention, a test portion of the hair coloring formulation (e.g. up to 25%, more preferably up to 15% and, more preferably up to 10%) is mixed, while it is extruded, from the applicator. This portion of the formulation is then applied to any part of the skin of the user as is known in the art. It will be appreciated that the amount that is withdrawn from the barrels of the syringe must be sufficient to fill the mixer nozzle and to provide an aliquot of the formulation sufficient for testing on the skin of the user. The applicator is preferably stored such that the components of the hair colouring formulation are not exposed to oxygen during the test period. Accordingly, the application may be sealed during the test period such as by placing a closure cap on the mixer nozzle or the barrels of the syringe, and/or the applicator may be placed in an oxygen impermeable container, e.g., a foil pouch. More preferably, the applicator is stored such that the components of the hair colouring formulation are not exposed to oxygen prior to the test portion of the formulation being applied to the skin of the user. Accordingly, the individual components of the hair coloring formulation will be able to be stored without any significant degradation while the user tests the formulation on the user's skin. Subsequently, if the user does not react to the formulation, the applicator may be unsealed and/or removed from the foil pouch and a further portion of the formulation, and preferably all of the remaining formulation, is mixed while extruded from the applicator. It will be appreciated that the user may apply the mixed formulation that is extruded from the applicator directly to their scalp, e.g., if an applicator brush is provided.

In a preferred aspect of this invention, a mixer nozzle is removably engagable with the applicator so as to be connectable in flow communication with the storage compartments. According to this aspect of the invention, when pressure is applied to the storage compartments to dispense the individual components from the storage compartments, the components will be mixed as they pass through the mixer nozzle. After a desired amount of the formulation has been dispensed, the mixer nozzle may then be removed and washed out. This permits any of the mixed formulation to be removed from the mixer nozzle so that the mixer nozzle is then ready for re-use. The applicator, e.g., the outlet of the mixer nozzle, and preferably the outlets from the storage compartment, may be sealed while the formulation is tested. If the user's skin accepts the formulation, then the cleaned mixer nozzle may subsequently be re-attached to the applicator and used to dispense an additional desired amount of the formulation, which is then used to treat the hair of the user.

In an alternate embodiment, two mixer nozzles may be provided. In this alternate embodiment, it is not necessary for a user to wash out any of the mixer nozzles. Instead, after use, each mixer nozzle may be discarded. In accordance with this alternate embodiment, the first mixer nozzle is removably engagable with the applicator. The second mixer nozzle may be engagable with the applicator but need not be removably engagable with the applicator.

In a further alternate embodiment, a single mixer nozzle may be used. In accordance with this embodiment, after a desired amount of the formulation has been dispensed, the mixer nozzle may then be stored for later use, e.g., within 48 hours, such as by placing a cap on the end of the mixer nozzle and/or placing the application in a foil pouch. If the user's skin accepts the formulation, then the applicator may be prepared for re-use (e.g. removing the applicator from a storage container and/or removing a closure cap) and then used to dispense an additional desired amount of the formulation, which is then used to treat the hair of the user. It will be appreciated that a portion of the formulation may be extruded from the mixer nozzle and discarded so as to remove portions of the formulation that may have oxidized during storage.

Therefore, in accordance with one aspect of this invention, there is provided a method of coloring hair with a hair coloring formulation that is prepared by mixing together at least first and second components using an applicator having at least one storage compartment for each component, the method comprising:
(a) mixing, while extruding from the applicator, some of each component to form a first amount of the hair coloring formulation;
(b) sealing the applicator;
(c) unsealing the applicator; and,
(d) mixing, while extruding from the applicator, additional amounts of each component to prepare an additional amount of the hair coloring formulation.

In one embodiment, a minor portion of the hair coloring formulation is mixed during step (a).

In another embodiment, less than 20 volume percent of the hair coloring formulation is mixed during step (a).

In another embodiment, the method further comprises applying at least some of the first amount to a user to determine if the user reacts to the hair coloring formulation and, after a sufficient amount of time has elapsed to determine if the user has a reaction to the hair coloring formulation and the user has no reaction, then conducting step (d) and using the additional amount of the hair coloring formulation to treat the hair of the user.

In another embodiment, each compartment has an outlet and steps (b) and (c) comprise covering the outlets and subsequently uncovering the outlets prior to conducting step (d).

In another embodiment, step (a) comprises mixing each component to form the hair coloring formulation while the components pass from the compartments through a first mixer nozzle that is in flow communication with the compartments. In accordance with this embodiment, the method may further comprise removing the first mixer nozzle from the applicator, installing a second mixer nozzle on the applicator and step (d) comprises mixing the components to form the additional amount of the hair coloring formulation while the components pass from the compartments through the second mixer nozzle that is in flow communication with the compartments. In accordance with this embodiment, each compartment may have an outlet and steps (b) and (c) may comprise covering the outlets after the first mixer nozzle has been removed and subsequently uncovering the outlets prior to step (d). In accordance with this embodiment, at least one of the mixer nozzles may have an openable valve and steps (b) and (c) may comprise using the valve to seal the outlets and subsequently opening the valve to uncover the outlets. In accordance with this embodiment, steps (b) and (c) may comprise installing a closure member after the first mixer nozzle is removed and subsequently removing the closure member prior to installing the second mixer nozzle on the applicator.

In another embodiment, each compartment has an outlet and the outlets are sealed when the applicator is acquired by a user and the method further comprises unsealing the outlets prior to step (a). In accordance with this embodiment, the method further comprises installing a first mixer nozzle on the applicator prior to conducting step (a).

In another embodiment, step (a) comprises passing the components through a mixer nozzle, and the method further comprises
(e) removing a mixer nozzle from the applicator;
(f) washing the hair coloring formulation from the mixer nozzle; and,
(g) reinstalling the mixer nozzle on the applicator prior to conducting step (d).

In another embodiment, each compartment has an outlet and the outlets are sealed when the applicator is acquired by a user and the method further comprises unsealing the outlets prior to step (a).

In another embodiment, the method further comprises installing the mixer nozzle on the applicator prior to conducting step (a).

In accordance with another aspect of the instant invention, there is provided a hair treatment kit for use in treating hair with a formulation prepared by mixing together at least first and second components, the kit comprising:
(a) an applicator having at least one compartment for each component and having an open end;
(b) at least one closure member positioned adjacent the open end of each compartment, whereby the closure member seals the open ends;
(c) a first dispenser nozzle releasably engagable with the applicator, whereby when engaged with the applicator, the first dispenser nozzle is in fluid flow communication with the open end of a compartment containing each compartment; and,
(d) a second dispenser nozzle engagable with the applicator, whereby when engaged with the applicator, the second dispenser nozzle is in fluid flow communication with the open end of a compartment containing each compartment.

In one embodiment, the closure member comprises a releasably engagable cap.

In another embodiment, at least one of the dispenser nozzles is a mixer nozzle. In accordance with this embodiment, at least one of the dispenser nozzles may have a fluid passage configured to combine the components in each compartment as the components flow through the nozzle. In accordance with this embodiment, at least one of the dispenser nozzles may have an upstream portion in flow communication with the open end of each compartment and configured to receive each component and a downstream portion configured to mix the components as the components flow through the downstream portion. In accordance with this embodiment, the flow passage may include a portion that extends in a spiral.

In another embodiment, each of the dispenser nozzles is a mixer nozzle.

In another embodiment, when combined, the formulation is a hair dye or hair tint.

In another embodiment, the hair treatment kit further comprises a brush applicator.

In another embodiment, the formulation is a two-component system and the applicator is a dual barreled syringe.

In an alternate embodiment, a kit may utilize two applicators wherein each applicator has two storage compartments for containing a different component of a hair treatment formulation. In accordance with this embodiment, each applicator may have the mixer nozzle integrally formed as part of the applicator or non-removably attached thereto. Alternately, each applicator may be adapted to have a mixer nozzle attached thereto. For example, a single mixer nozzle may be provided with the two applicators. The mixer nozzle may first be applied to one applicator. After the hair coloring formulation has been dispensed from the first applicator, the mixer nozzle may then be cleaned so as to remove the hair coloring formulation therefrom. The mixer nozzle may then be attached to the second applicator. It will be appreciated that in accordance with this embodiment, the mixer nozzle is removably attachable to the first applicator but need not be removably attachable to the second applicator.

In accordance with a second embodiment, two mixer nozzles may be provided. Accordingly, it is not necessary for a user to clean a mixer nozzle between uses. After use, each mixer nozzle may be discarded. In accordance with this embodiment, it will be appreciated that the mixer nozzles may be removably attachable to the applicator or may be integrally formed as part of the applicator. If the mixer nozzles are removably attachable, then they may be configured so as to open each storage compartment when attached to a nozzle (e.g. they may have inlets which pierce the seal of the outlets of the storage compartments of the applicator). It will also be appreciated that if the mixer nozzle is formed as part of the applicator and is not removable therefrom, that a means must be provided to open the storage compartments. This opening means could comprise a valve which is positioned adjacent the outlets from the storage compartment and which is movable between an open and a closed position, a pull tab to remove a foil closure on the outlets from the storage compartment, an arm member with a piercing end which is movably mounted with respect to the outlets of the storage compartments and which is actuated by a lever on the exterior of the applicator, or any other means known in the art.

Therefore, in accordance with another aspect of the instant invention, there is provided a method a coloring hair using two applicators wherein the hair coloring formulation is prepared by mixing together at least first and second components and wherein each applicator has at least one storage compartment for each component, the method comprising:
(a) mixing, while extruding from the first applicator, at least some of each component to form a first amount of the hair coloring formulation; and,
(b) mixing, while extruding from the second applicator, at least some of each component to prepare an additional amount of the hair coloring formulation.

In one embodiment, the method further comprises applying at least some of the first amount to a user to determine if the user reacts to the hair coloring formulation and, after a sufficient amount of time has elapsed to determine if the user has a reaction to the hair coloring formulation and the user has no reaction, then conducting step (b) and using the additional amount of the hair coloring formulation to treat the hair of the user.

In another embodiment, each compartment has an outlet and the outlets are sealed when the applicators are acquired by a user, and the method further comprises unsealing the outlets of the first applicator prior to step (a) and unsealing the outlets of the second applicator prior to step (b).

In accordance with another aspect of the instant invention, there is provided a hair treatment kit for use in treating hair with a formulation prepared by mixing at least first and second components together, the kit comprising:
(a) a first applicator having at least one compartment for each component and each compartment has an open end;
(b) a first closure member positioned adjacent the open end of each compartment of the first applicator, whereby the closure member seals the open ends;
(c) a first dispenser nozzle engagable with the first applicator, whereby when engaged with the first applicator, the first dispenser nozzle is in fluid flow communication the open end of a compartment containing each compartment;
(d) a second applicator having at least one compartment for each component and each compartment has an open end; and,
(e) a second closure member positioned adjacent the open end of each compartment of the second applicator, whereby the closure member seals the open ends.

In one embodiment, the hair treatment kit further comprises a second dispenser nozzle engagable with the second applicator, whereby when engaged with the second applicator, the second dispenser nozzle is in fluid flow communication with the open end of a compartment containing each compartment.

In another embodiment, the first applicator has a smaller storage volume then the second applicator.

In another embodiment, the first dispenser nozzles is a mixer nozzle.

In another embodiment, when combined, the formulation is a hair dye or hair tint.

In another embodiment, the hair treatment kit further comprises a brush applicator.

In another embodiment, the formulation is a two-component system and each applicator is a dual barreled syringe.

In another embodiment, the applicator has an outlet end and step (b) comprises placing a cap on the outlet end.

In another embodiment, the applicator is packaged in a sealed air impermeable container and the method further comprises opening the package prior to step (a).

In another embodiment, the method further comprises providing the applicator and a gas having an oxygen reduced content in the sealed air impermeable container.

In another embodiment, the method further comprises subjecting the air impermeable container to a nitrogen gas purge to obtain the gas having an oxygen reduced content.

In another embodiment, the method further comprises subjecting the applicator a nitrogen gas purge and subsequently filling the applicator with the components prior to step (a) of claim 1.

In accordance with another embodiment of the instant invention, there is provided a method of coloring hair with a hair coloring formulation that is prepared by mixing together at least first and second components using an applicator having at least one storage compartment for each component, the method comprising:
(a) subjecting the applicator a nitrogen gas purge;
(b) subsequently filling the applicator with the components; and
(c) extruding from the applicator, some of each component to form a first amount of the hair coloring formulation.

In one embodiment, the method further comprises providing the applicator in a sealed air impermeable container.

In another embodiment, the method further comprises providing the applicator and a gas having an oxygen reduced content in the sealed air impermeable container.

In another embodiment, the method further comprises subjecting the air impermeable container to a nitrogen gas purge to obtain the gas having an oxygen reduced content.

In another embodiment, the method further comprises storing the applicator by one or more of placing a closure member on the applicator and placing the applicator in an air impermeable container before subsequently extruding from the applicator additional amounts of each component.

In another embodiment, the method further comprises waiting to determine if the skin of a user reacts to the hair colouring formulation prior to extruding from the applicator additional amounts of each component.

One advantage of such kits and applicators is that the user may dispense only a portion of the formulation that is provided and store the remainder for after the test period. If the user does not react to the formulation, then the remainder of the formulation may be dispensed. A further advantage is that the formulation may be automatically mixed as it is dispensed. A further advantage with some aspects of this invention is that the user need not clean the dispenser between uses but may merely dispose of a dispenser and utilize a clean dispenser, which is provided as part of the kit.

### BRIEF DESCRIPTION OF THE DRAWINGS

These and other advantages of the instant invention will be more fully and clearly understood in connection with the following description of the preferred embodiments of the invention in which:

FIG. 1 is a partially cut away perspective view of a kit according to a first embodiment of the instant invention;

FIG. 2 is a perspective view of the kit of FIG. 1 in use;

FIG. 3 is a partially cut away perspective view of a kit according to an alternate embodiment of the instant invention;

FIG. 4 is a cut away perspective view of a kit according to a further alternate embodiment of the instant invention;

FIG. 5 is a perspective view of a mixing nozzle being attached to the dual barreled syringe shown in FIG. 1;

FIG. 6 is an enlarged view showing the mixer nozzle attached to the dual barreled syringe of FIG. 1;

FIG. 7 is a perspective view of the closure cap when removed from the end of the dual barreled syringe of FIG. 1;

FIG. 8 is an end view of the closure cap of FIG. 7;

FIG. 9 is an end view of the dual barreled syringe of FIG. 7; and,

FIG. 10 is a cross section through the closure cap and the dual barreled syringe along the line 10-10 shown in FIG. 1.

### DETAILED DESCRIPTION OF THE INVENTION

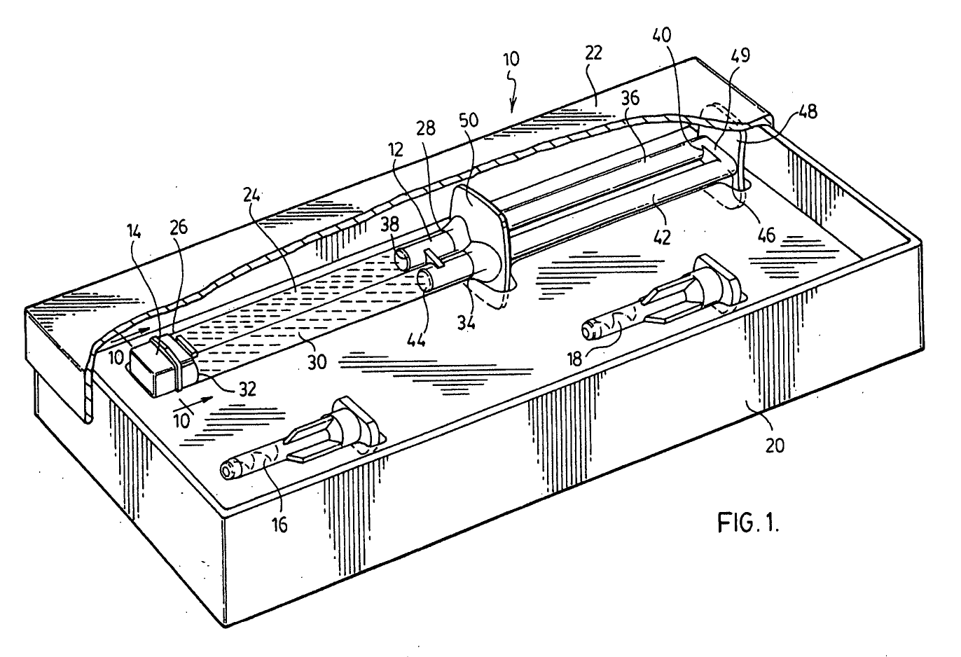

As shown in FIG. 1, a kit 10 according to one preferred embodiment of the instant invention comprises an applicator 12, a closure member 14 and first and second dispenser nozzles 16 and 18.

The kit may be provided in any packaging and, preferably, a packaging suitable for retail sale. For example, as shown in FIG. 1, the kit 10 is provided in a package having a bottom 20 that is provided with recesses for removably receiving therein the components of the kit, and a top 22, a portion of which is broken away in FIG. 1 so as to reveal the contents of the container. It will be appreciated that all or a portion of top 22 may be transparent (e.g. it could be made from thin plastic). It will also be appreciated that the packaging could be a blister pack or other package known in the art.

Applicator 12 may be any applicator known in the art, which has at least as many compartments as the formulation to be mixed. Preferably, the formulation is a two-component system and applicator 12 has two storage compartments—one for each component. For example, applicator 12 may have two storage compartments wherein the contents of each of the storage compartments is emptied at the same time when a user actuates a dispenser or squeezes on the storage compartments.

In one embodiment, the storage compartments may be individually formed and then assembled into a frame so as to form a single applicator 12. In another embodiment, the dispenser nozzle itself may provide the frame or holder for receiving individually formed storage compartments. Most preferably, applicator 12 comprises a dual barreled syringe.

As shown in FIG. 1, a dual barreled syringe has a first storage compartment or barrel 24 having a first end 26 and a second end 28 and a second compartment or barrel 30 having a first end 32 and a second end 34. Applicator 12 also includes a first plunger 36 having a first end 38 and a second end 40, and a second plunger 42 having a first end 44 and a second end 46. Plungers 36, 42 may be connected together so that each plunger is actuated at the same time. Accordingly, a cross member 49 and/or a first flange 48 may connect the second ends 40, 46 of plungers 36, 42 together. In order to secure the barrels 24, 30 together, a second flange 50 may be provided. Second flange 50 may be secured to send ends 28, 34 of barrels 24, 30. Accordingly, when assembled together, barrels 24, 30, plungers 36, 42 and flanges 48, 50 may create a one-piece assembly. It will be appreciated that barrels 24, 30 may be co-extruded so as to be integrally formed. Similarly, plungers 36, 42 may be integrally formed with cross member 49, and optionally together with flanges 48 and/or 50. Any other construction technique known in the art may be used.

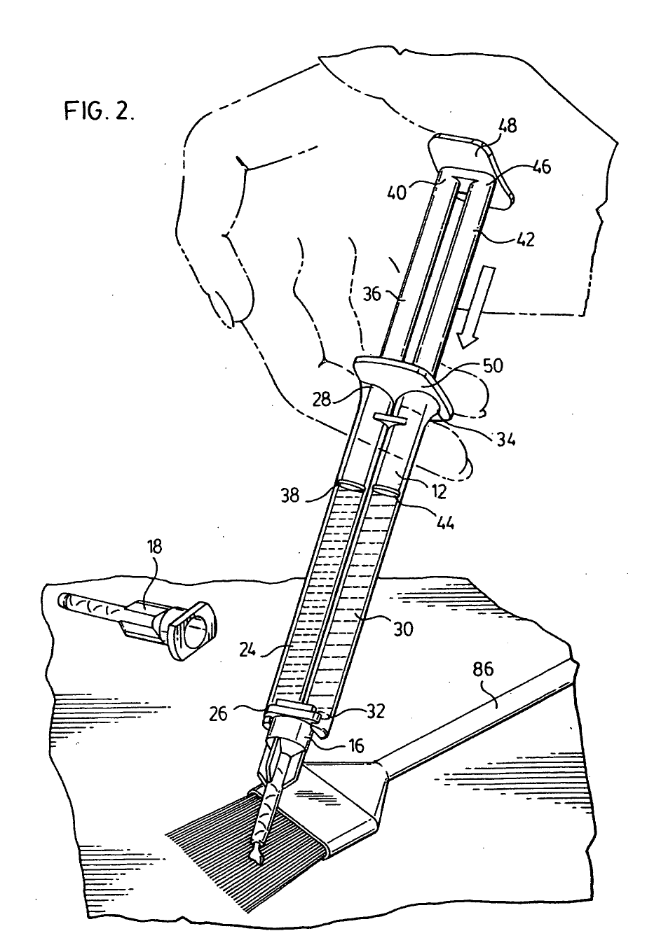

In operation, a user may grasp flanges 48 and 50 as shown in FIG. 2 and apply pressure to flange 48 so as to force plungers 36, 42 to pass through barrels 24, 30 thereby dispensing the components in barrels 24, 30.

If the formulation is an oxidative hair coloring formulation, then the dye component may be provided in barrel 24 and the developer may be provided in barrel 30. The dye component and developer may be designed to be mixed in a one to one ratio. As such, barrels 24, 30 may have the same volume and may be dispensed at the same rate. It will also be appreciated that if the dye component and developer are to be mixed in a different ratio, that applicator 12 could be configured to dispense the developer and the dye component at different rates so that the dye component and developer are dispensed in the required ratio. It will also be appreciated that the volume of the barrels may differ, such as by varying the length and/or diameter of barrels 24, 30.

Dispenser nozzles 16, 18 are adapted to be attached to dual barreled syringe 12. It will be appreciated that in accordance with one embodiment of this invention, dispenser nozzle 16 may be removably attachable to dual barrel syringe 12 and dispenser nozzle 18 may be non-removably attachable to dual barreled syringe 12 (i.e. once attached to dual barreled syringe 12, it is not designed to be removed thereform). Preferably, each dispenser nozzle 16, 18 is removably attachable to dual barreled syringe 12.

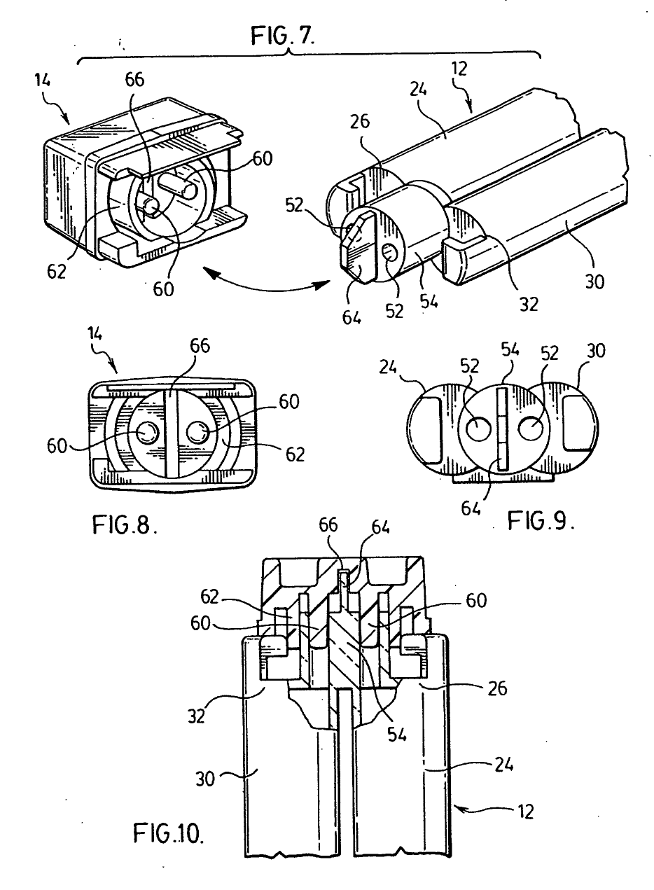

Each of barrels 24, 30 has an outlet 52. For example, as shown in FIGS. 7 and 9, first ends 26, 32 of barrels 24, 30 are each provided with at least one outlet 52. As shown therein, outlets 52 extend through housing 54 so as to be in fluid flow communication with the interior of barrels 24, 30. It will be appreciated that outlets 52 may be of any particular configuration and may be provided directly on ends 26, 32 of barrels 24, 30.

When dual barreled syringe 12 is acquired by a user, dual barreled syringe 12 is preferably sealed and, more preferably, outlets 52 are sealed. It will be appreciated that any member which seals applicator 12 and, preferably, outlets 52 to prevent the dye component and developer from mixing while still in applicator 12 and to prevent degradation of the dye component and the developer by exposure to the atmosphere may be utilized. For example, as shown in FIG. 1, dual barreled syringe 12 may be provided with a closure member 14 that is removably attachable to dual barreled syringe 12. Alternately, as shown in FIG. 4, dual barreled syringe 12 may be provided in kit 10 with a foil closure member 16 having a pull-tab 58. A user may grasp pull-tab 58 to remove foil closure cap 56 to open dual barreled applicator 12. Any such closure member known in the art may be used. For example, the closure member need not be made from foil but may be made from plastic or a water impermeable cellulose substrate. It will be appreciated that this form of closure member is not re-usable. If a disposable closure member is used (e.g., closure member 56), then a closure member 14 that is removably attachable to dual barreled syringe 12 is preferably provided if kit 10 includes a single dual barreled syringe 12. However, if kit 10 comprises two applicators 12, each of which is sealed by a disposable closure member is used (e.g., closure member 56), then kit 10 need not comprise a closure member 14 that is removably attachable to dual barreled syringe 12.

It will be appreciated that the interior of dual barreled syringe 12 may be purged with nitrogen prior to dual barreled syringe being filled and, in addition, the container for the kit. In such a case, dual barreled syringe may optionally also be sealed as well as being stored in a nitrogen environment.

FIGS. 7 and 8 show a preferred embodiment of a resealable cap 14. As shown therein, the rear face of closure cap 14 has two sealing members 60. Each sealing member 60 is adapted to be received in an outlet 52 thereby sealing outlets 52. In order to secure closure member 14 to dual barreled syringe 12, the rear face of closure member 14 may be provided within annular member 62 that surrounds outlets 60. Annular member 62 is preferably sized so as to be slidingly received on the outer surface of housing 54. Frictional engagement between the inner wall of annular member 62 and the outer wall of housing 54 may be used to maintain closure member 14 secured to housing 54. Alternately, or in addition, other securing means know in the art may be used such as male and female engagement members, one of which is provided on closure member 14 and the other of which is provided on syringe 12. Optionally an alignment member 64 may be provided on housing 54 and an opening or slot 66 may be provided in closure members 14 for removably receiving alignment member 64. Optional alignment member 64 aids in guiding sealing members 60 into outlets 52 as closure member 14 is provided on housing 54.

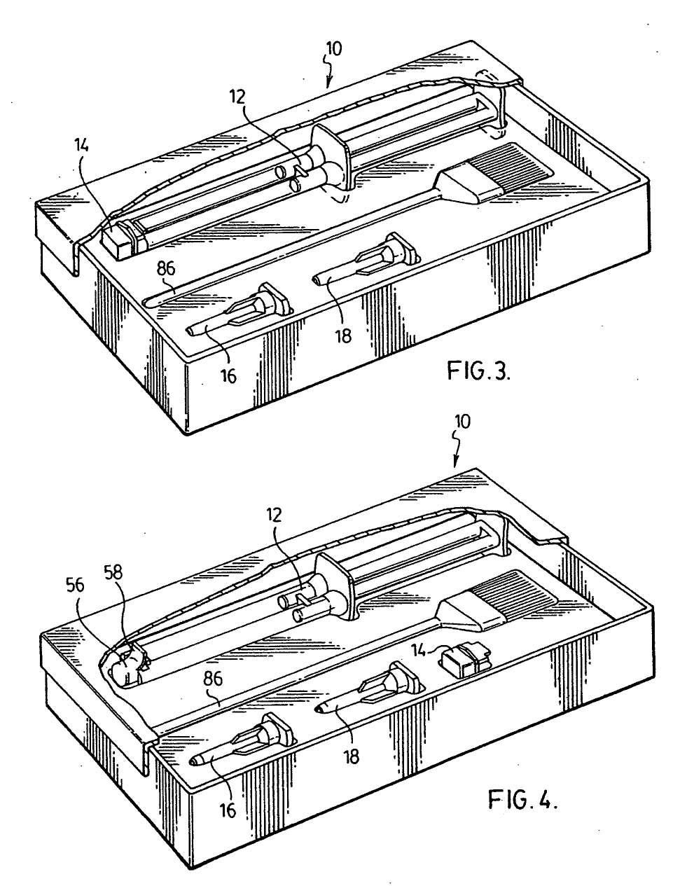

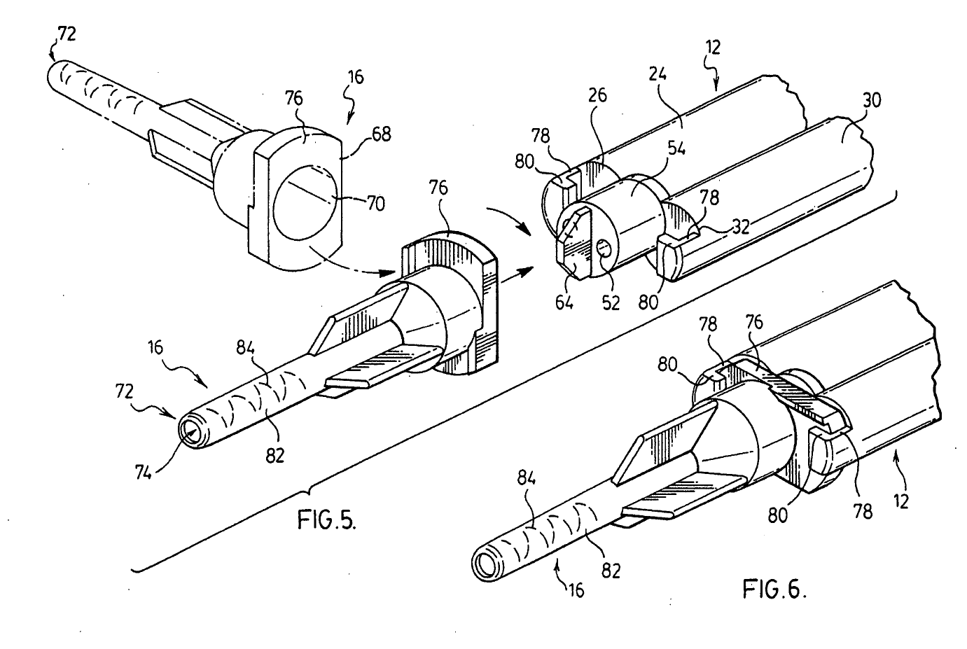

Dispenser nozzles 16, 18 preferably are configured to mix the dye component and developer together as they flow through dispenser nozzle 16, 18. Accordingly, dispenser nozzles 16, 18 are preferably mixer nozzles. As shown in FIGS. 5 and 6, nozzles 16, 18 preferably have an inlet end 68 having an opening 70 sized to receive at least a portion of housing 54, an outlet end 72 having at least one outlet 74 and a mixer portion 82, which is configured to at least partially mix the components together. For example, mixer nozzle 82 may have an extended flow passage that may be in the form of a helical passage 84. A moveable mechanical member may be provided to assist in mixing the components together as they pass through nozzle 16, 28. More preferably, the formulation is at least essentially mixed when it is dispensed from nozzle 16, 18. By “at least essentially mixed”, it is meant that the formulation is mixed sufficiently so that it may be used without further mixing.

It will be appreciated that outlet end 72 may have a hollow comb or brush applicator receivably mountable on the end thereof or integrally formed thereon. Any applicator comb or brush, which is designed to have a fluid flow therethrough, may be provided on or with nozzle 16, 18. Preferably, two such applicator comb or brushes are provided.

Nozzle 16, 18 may be receivably mounted on dual barreled syringe 12 by any means known in the art. For example, as shown in FIG. 5, inlet end 68 of nozzle 16, 18 is provided with a flange 76. Dual barreled syringe 12 is provided with flanges 78, the distal end of each of which has an arm member 80 provided thereon. Accordingly, nozzle 16, 18 may be aligned with housing 54 as shown in FIG. 5 and then moved towards housing 54 so as to receive housing 54 in opening 70. Subsequently, nozzle 16, 18 may be rotated 90° relative to dual barreled syringe 12 to the position shown in FIG. 6 such that arms 80 engage flange 76. Accordingly, nozzle 16, 18 is removably secured to dual barreled syringe 12. It will be appreciated that nozzle 16, 18 may be engagably secured and, preferably, releasably secured to dual barrel syringe 12 by any other means known in the art.

It will also be appreciated that, in accordance with another embodiment of the instant invention, the applicator may have a nozzle 16 provided thereon when the applicator is purchased by a user. Nozzle 16 may be permanently attached to the applicator or removable mounted thereon. In such a case, the syringe with applicator attached may be sold in a package that contains only the pre-filled applicator with nozzle 16 attached. In accordance with such an embodiment, a user need not have to assemble the application but may merely use a preassembled application.

Preferable, the applicator or kit is provided in a package, which was subjected to a purge to remove or reduce the oxygen in the package, prior to the package being sealed.

In use, the components in barrels 24, 30 are mixed as they flow through nozzles 16, 18. In the embodiment shown in FIGS. 5 and 6, when the components are dispensed from barrels 24, 30 via outlets 52, the components enter mixer portion 82. Accordingly, when the components exit outlet 74, the dye component and developer are preferably essentially mixed (i.e. preferably no additional mixing is required prior to use of the hair coloring formulation).

The use of kit 10 will now be discussed. In the embodiment of FIG. 1, kit 10 includes a single applicator 12. When a user acquires kit 10, outlets 52 of barrels 24, 30 are preferably sealed, such as by removable closure member 14. Further, kit 10 may be in an oxygen impermeable container. The user will open the oxygen impermeable container, if provided, and/or remove closure member 14 (e.g. by pulling closure member 14 off of housing 52) as may be required. First dispenser nozzle 16 may then be secured to applicator 12 such as by the mechanism shown in FIGS. 5 and 6. The user may then force plungers 36, 42 partially into barrels 24, 30 so as to mix while dispensing a first aliquot or amount (e.g., 20 volume percent or less) of the hair coloring formulation. The formulation may be dispensed onto a brush or other surface, or directly on to some of the hair or skin of the user. Preferably, a small amount of the formulation is dispensed and allowed to remain in contact with the skin of the user for a sufficient amount of time to determine if the scalp of the user will react to the hair coloring formulation. After the first aliquot of formulation has been dispensed, nozzle 16 is preferably removed and discarded. Closure member 14 is then re-applied to dual barreled syringe 12 to seal barrels 24, 30; Subsequently, e.g., after a sufficient amount of time has passed for a user to determine if the skin of the user reacts to the hair coloring formulation, the user may again remove closure member 14 and install second dispenser nozzle 18. The user may then dispense an additional amount of the hair coloring formulation and use the hair coloring formulation as needed.

In accordance with an alternate method of the instant embodiment, it will be appreciated that instead of providing two dispenser nozzles 16, 18 that a kit 10 may contain a single removably mountable dispenser nozzle 16. In accordance with this embodiment of the invention, after the first aliquot of hair coloring formulation has been dispensed, the user cleans applicator 16 to remove the hair coloring formulation therein. Accordingly, if the skin of the user does not react to the hair coloring formulation, the user may remove closure member 14 and re-install dispenser nozzle 16. It will also be appreciated that this method may be used with the kit of FIG. 1.

In accordance with the alternate embodiment shown in FIG. 3 of the instant invention, a kit 10 may comprise an applicator 12, two closure members 14 and two nozzles 16, 18. Brush 86 may optionally be provided. In accordance with this embodiment of the invention, after the oxygen impermeable container, if provided, is opened, a user may remove closure member 14 from applicator 12 and discard closure member 14. First dispenser nozzle 16 may then be attached to applicator 12 and a first aliquot of the hair coloring formulation dispensed. Nozzle 16 may then be discarded and second closure member 14 affixed to applicator 12. Subsequently, the second closure member 14 may be removed and second dispenser nozzle 18 installed and additional amounts of the hair coloring formulation may be dispensed. The provision of a second closure member may be advantageous to prevent or reduce the likelihood of some of the dye component being contaminated with developer while in applicator 12. For example, if some of the developer is on a sealing member 60 or is within the opening defined by annular member 62, then some of the developer may be inserted into the outlet 52 of the dye component when closure cap 14 is re-applied to applicator 12. The use of a clean closure member 14 will prevent such contamination.

In accordance with the alternate embodiment shown in FIG. 4, kit 10 comprises an applicator 12, a foil closure member 56, two nozzles 16, 18 and a removably attachable closure member 18. An optional brush 86 may be provided. In accordance with this embodiment, the method comprises opening the oxygen impermeable container, if provided, removing and disposing of closure member 56 and attaching dispenser nozzle 16 to applicator 12. After a first aliquot of the hair coloring formulation has been dispensed, nozzle 16 is removed and discarded and closure member 14 is applied to applicator 12. Subsequently, closure member 14 may be removed and second dispenser nozzle 18 installed on applicator 12. Further amounts of the hair coloring formulation may then be dispensed. In a variation of this method, it will be appreciated that kit 10 of FIG. 4 may comprise a single applicator 16 and, accordingly, a user may wash out applicator 16 prior to the re-use of applicator 16.

In accordance with a further alternate embodiment of the instant invention, it will be appreciated that a kit 10 may comprise two applicators 12, each of which is sealed, and at least one dispenser nozzle 16 and, preferably, two dispenser nozzles 16, 18. One or both of the applicators 12 may be sealed with a removable closure member 14 or a foil closure member 56. In accordance with such an embodiment, a user may open the oxygen impermeable container, if provided and then take a first applicator 12, remove the closure member and install a nozzle 16. The user may then dispense an aliquot of the hair coloring formulation for testing purposes. The applicator 12 may then be disposed of with or without nozzle 16. If the applicator 12 is disposed of with nozzle 16, then kit 10 is provided with a second nozzle 18. After the test period, the user may then take the second applicator 12, remove the closure member and install the second nozzle 16 (or a washed first nozzle 16). Additional amounts of hair coloring formulation may then be dispensed for use.

It will be appreciated that since each applicator 12 is disposed of after use, kit 10 may include two nozzles 16, 18 that need not be removably mountable on applicators 12. For example, nozzles 16, 18 may be non-removably engagable with applicator 12, provided already installed on one or each applicator 12 or integrally formed as part of each applicator 12. If nozzles are provided already installed or integrally formed as part of applicators 12, then a means is provide to permit outlets 52 to be opened. For example, each nozzle 16, 18 may have a valve formed therein. Alternately, a foil closure member 56 may be provided to cover outlets 52 and a pull-tab 58 may extend out beyond nozzle 16. Any other opening means known in the art may be used.

In accordance with a further alternate embodiment of the instant invention, it will be appreciated that a kit 10 may comprise a single applicator F 12 which has a nozzle 16 attached thereto. Kit 10 may sealed, such as by providing application 12 and nozzle 16 in an air impermeable container (e.g., a foil pouch. Alternately, or in addition, and preferably in addition, nozzle 16 is sealed, such as by a closure member on the end of nozzle 16 distal to barrels 24, 30.

The user may then dispense an aliquot of the hair coloring formulation for testing purposes. The applicator 12 and nozzle 16 may then be stored, such as by being sealed in the air impermeable container, which is preferably resealable by any means known in the art, and/or by placing a cap on the end of nozzle 16. After the test period, the user may then take the applicator 12, remove the cap and/or open the air impermeable container and dispense additional amounts of hair coloring formulation.

In accordance with this embodiment, applicators 12 may contain different amounts of the hair coloring formulation. In particular, one of the applicators 12 may contain only a sufficient amount of the hair coloring formulation to permit the user to test the formulation on the skin of the user. The other applicator 12 may contain the required amount to permit the user to color their hair. Accordingly, one of the applicators may contain about 20%, and, more preferably, about 10% of the amount of the hair coloring formulation in the other applicator.

It will be appreciated by those skilled in the art that various additions and modifications may be made to the kit and the method disclosed herein and all such modifications and additions are within the scope of the following claims. In particular, the method of attachment of nozzle 16, 18 to applicator 12, the method of attachment of closure members 14, 56, the construction of the nozzles, the applicator, the closure members or the brush may be any of those known in the art. It will also be appreciated that the hair treatment formulation may be any two-component dye, tint or other hair treatment known in the art. In addition, the packaging for kit 10 may be of any design.

1. A method a coloring hair with a hair coloring formulation that is prepared by mixing together at least first and second components using an applicator having at least one storage compartment for each component, the method comprising:
a) mixing, while extruding from the applicator, some of each component to form a first amount of the hair coloring formulation;
b) sealing the applicator;
c) unsealing the applicator; and,
d) mixing, while extruding from the applicator, additional amounts of each component to prepare an additional amount of the hair coloring formulation.

2. The method as claimed in claim 1 wherein a minor portion of the hair coloring formulation is mixed during step (a).

3. The method as claimed in claim 1 wherein less than 20 volume percent of the hair coloring formulation is mixed during step (a).

4. The method as claimed in claim 1 further comprising applying at least some of the first amount to a user to determine if the user reacts to the hair coloring formulation and, after a sufficient amount of time has elapsed to determine if the user has a reaction to the hair coloring formulation and the user has no reaction, then conducting step (d) and using the additional amount of the hair coloring formulation to treat the hair of the user.

5. The method as claimed in claim 1 wherein each compartment has an outlet and steps (b) and (c) of claim 1 comprise covering the outlets and subsequently uncovering the outlets prior to conducting step (d).

6. The method as claimed in claim 1 wherein step (a) comprises mixing each component to form the hair coloring formulation while the components pass from the compartments through a first mixer nozzle that is in flow communication with the compartments.

7. The method as claimed in claim 6 further comprising removing the first mixer nozzle from the applicator, installing a second mixer nozzle on the applicator and step (d) comprises mixing the components to form the additional amount of the hair coloring formulation while the components pass from the compartments through the second mixer nozzle that is in flow communication with the compartments.

8. The method as claimed in claim 7 wherein each compartment has an outlet and steps (b) and (c) of claim 1 comprises covering the outlets after the first mixer nozzle has been removed and subsequently uncovering the outlets prior to step (d).

9. The method as claimed in claim 8 wherein at least one of the mixer nozzles has an openable valve and steps (b) and (c) of claim 1 comprise using the valve to seal the outlets and subsequently opening the valve to uncover the outlets.

10. The method as claimed in claim 9 wherein steps (b) and (c) of claim 1 comprise installing a closure member after the first mixer nozzle is removed and subsequently removing the closure member prior to installing the second mixer nozzle on the applicator.

11. The method as claimed in claim 1 wherein each compartment has an outlet and the outlets are sealed when the applicator is acquired by a user and the method further comprises unsealing the outlets prior to step (a) of claim 1.

12. The method as claimed in claim 11 further comprising installing a first mixer nozzle on the applicator prior to conducting step (a) of claim 1.

13. The method as claimed in claim 1 wherein step (a) comprises passing the components through a mixer nozzle, and the method further comprises:
e) removing a mixer nozzle from the applicator;
f) washing the hair coloring formulation from the mixer nozzle; and,
g) reinstalling the mixer nozzle on the applicator prior to conducting step (d) of claim 1.

14. The method as claimed in claim 13 wherein each compartment has an outlet and the outlets are sealed when the applicator is acquired by a user and the method further comprises unsealing the outlets prior to step (a) of claim 1.

15. The method as claimed in claim 14 further comprising installing the mixer nozzle on the applicator prior to conducting step (a) of claim 1.

16. The method as claimed in claim 1 wherein the applicator has an outlet end and step (b) comprises placing a cap on the outlet end.

17. The method as claimed in claim 1 wherein the applicator is packaged in a sealed air impermeable container and the method further comprises opening the package prior to step (a).

18. The method as claimed in claim 17 further comprising providing the applicator and a gas having an oxygen reduced content in the sealed air impermeable container.

19. The method as claimed in claim 17 further comprising subjecting the air impermeable container to a nitrogen gas purge to obtain the gas having an oxygen reduced content.

20. The method as claimed in claim 16 further comprising subjecting the applicator a nitrogen gas purge and subsequently filling the applicator with the components prior to step (a) of claim 1.

21. A method a coloring hair with a hair coloring formulation that is prepared by mixing together at least first and second components using an applicator having at least one storage compartment for each component, the method comprising:
a) subjecting the applicator a nitrogen gas purge;
b) subsequently filling the applicator with the components; and
c) extruding from the applicator, some of each component to form a first amount of the hair coloring formulation.

22. The method as claimed in claim 21 further comprising providing the applicator in a sealed air impermeable container.

23. The method as claimed in claim 22 further comprising providing the applicator and a gas having an oxygen reduced content in the sealed air impermeable container.

24. The method as claimed in claim 23 further comprising subjecting the air impermeable container to a nitrogen gas purge to obtain the gas having an oxygen reduced content.

25. The method as claimed in claim 21 further comprising storing the applicator by one or more of placing a closure member on the applicator and placing the applicator in an air impermeable container before subsequently extruding from the applicator additional amounts of each component.

26. The method as claimed in claim 21 further comprising waiting to determine if the skin of a user reacts to the hair colouring formulation prior to extruding from the applicator additional amounts of each component.
